(19) United States
(12) Patent Application Publication
(10) Pub. No.: US 2024/0374866 A1
(43) Pub. Date: Nov. 14, 2024

(54) RAPID EXCHANGE SYSTEM FOR OTW DELIVERY

(71) Applicant: Boston Scientific Scimed, Inc., Maple Grove, MN (US)

(72) Inventors: Joel T. Eggert, Plymouth, MN (US); James P. Rohl, Prescott, WI (US); Aaron Abbott, Columbia Heights, MN (US); Daniel Shuey, Pine City, MN (US); James K. Cawthra, JR., Ramsey, MN (US); Joseph Barrett Fitzgerald, Edina, MN (US)

(73) Assignee: Boston Scientific Scimed, Inc., Maple Grove, MN (US)

(21) Appl. No.: 18/656,790

(22) Filed: May 7, 2024

Related U.S. Application Data
(60) Provisional application No. 63/464,751, filed on May 8, 2023.

(51) Int. Cl. A61M 25/00; A61M 25/01; A61M 25/06; A61M 25/09; A61M 39/02; A61M 39/06

(52) U.S. Cl. CPC A61M 25/0029; A61M 25/0097; A61M 25/09; A61M 39/06; A61M 2025/0183; A61M 2025/0681; A61M 2039/0258; A61M 2039/0264; A61M 2039/0273; A61M 2039/062

(57) ABSTRACT

A medical system includes an introducer device having an elongate shaft defining a shaft lumen and a hub secured to a proximal region of the elongate shaft. The hub includes a primary channel extending through the hub and fluidly coupled with the shaft lumen and a secondary channel extending through the hub and fluidly coupled with the shaft lumen. A guide catheter is adapted to be advanced through the secondary channel and into the shaft lumen. A guidewire is adapted to be advanced through the secondary channel and into the shaft lumen. A working catheter is adapted to be advanced through the primary channel and into the shaft lumen when the guide catheter is positioned with a distal end of the guide catheter within the secondary channel, the working catheter including a catheter shaft including a distal region that is adapted to releasably engage the guidewire.

### CROSS-REFERENCE TO RELATED APPLICATIONS

This application claims the benefit of priority under 35 U.S.C. § 119 of U.S. Provisional Application No. 63/464,751, filed May 8, 2023, the entire disclosure of which is hereby incorporated by reference.

### TECHNICAL FIELD

The disclosure is directed to an introducer. More particularly, the disclosure is directed to an introducer that keeps OTW tools within a saline flow.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and the use thereof. An example may be found in a medical system including an introducer device including an elongate shaft defining a shaft lumen therethrough, the elongate shaft including a proximal region, and a proximal hub secured relative to the proximal region of the elongate shaft. The proximal hub includes a primary channel extending through the proximal hub and fluidly coupled with the shaft lumen and a secondary channel extending through the proximal hub and fluidly coupled with the shaft lumen. A guide catheter is adapted to be advanced through the secondary channel and into the shaft lumen. A guidewire is adapted to be advanced through the secondary channel. A working catheter is adapted to be advanced through the primary channel and into the shaft lumen when the guide catheter has been withdrawn proximally such that a distal end of the guide catheter remains within the secondary channel, the working catheter including a catheter shaft including a distal region, the distal region of the working catheter adapted to releasably engage the guidewire.

Alternatively or additionally, the medical system may further include a first hemostasis valve fluidly coupled with the primary channel and a second hemostasis valve fluidly coupled with the secondary channel.

Alternatively or additionally, the medical system may further include a third hemostasis valve disposed between the proximal hub and the elongate shaft, thereby providing a fluid tight volume within the primary channel and the secondary channel.

Alternatively or additionally, the guidewire may have a first diameter over the guidewire outside of an engagement region, and a second diameter less than the first diameter within the engagement region.

Alternatively or additionally, the distal end of the working catheter may include a distal body defining a primary engagement feature adapted to releasably engage the guidewire.

Alternatively or additionally, the primary engagement feature may include a body lumen having a body lumen diameter and a channel operably coupled with the body lumen, the channel having a channel width less than the body lumen diameter.

Alternatively or additionally, the channel width may be less than the first diameter of the guidewire and greater than the second diameter of the guidewire, and the body lumen diameter may be greater than the first diameter of the guidewire.

Alternatively or additionally, the engagement feature may further include one or more secondary engagement features disposed proximally of the primary engagement feature.

Alternatively or additionally, each of the one or more secondary engagement features may include C-shaped features having a lumen diameter greater than the first diameter of the guidewire and a channel width that is greater than the second diameter of the guidewire.

Alternatively or additionally, the primary channel may be curved and the secondary channel may be straight and may intersect the primary channel.

Another example may be found in an introducer device for use with a guide catheter and a working catheter. The introducer device includes an elongate shaft defining a shaft lumen therethrough and a hub secured relative to a proximal region of the elongate shaft. The hub includes a first hemostasis valve disposed at a proximal end of the hub, a second hemostasis valve disposed at the proximal end of the hub, the second hemostasis valve parallel with but spaced from the first hemostasis valve, a primary channel extending distally through the hub from the first hemostasis valve to the shaft lumen and a secondary channel extending distally through the hub from the second hemostasis valve and fluidly coupled with the primary channel.

Alternatively or additionally, the primary channel may curve through the hub from the first hemostasis valve towards the shaft lumen.

Alternatively or additionally, the secondary channel may extend linearly through the hub from the second hemostasis valve to an intersection between the primary channel and the secondary channel.

Alternatively or additionally, a diameter of the secondary channel may narrow from proximal to distal.

Alternatively or additionally, the introducer device may further include a third hemostasis valve fluidly coupled between the primary channel and the shaft lumen, thereby providing a fluid-tight volume between the first hemostasis valve, the second hemostasis valve and the third hemostasis valve.

Another example may be found in a working catheter adapted to be used in combination with an introducer device having a bifurcated hub, the bifurcated hub defining a first passageway adapted to accommodate the working catheter and a second passageway adapted to accommodate a guide catheter and a guidewire, the second passageway intersecting the first passageway, the guidewire having a first diameter outside of an engagement region and a second diameter less than the first diameter within the engagement region. The working catheter includes an elongate catheter shaft extending from a proximal region to a distal region and a working member secured within the distal region. The working member includes a primary engagement feature that is adapted to releasably secure the body member to a guidewire and one or more secondary engagement features that are each adapted to releasably secure the body member to the guidewire.

Alternatively or additionally, the working member may further include a working feature.

Alternatively or additionally, the primary engagement feature may include a body lumen having a body lumen diameter that is greater than the first diameter of the guidewire and a channel operably coupled with the body lumen, the channel having a channel width that is less than the first diameter of the guidewire and greater than the second diameter of the guidewire.

Alternatively or additionally, the channel width may be less than a first diameter of the guidewire and greater than a second diameter of the guidewire, and the body lumen diameter may be greater than the first diameter of the guidewire.

Alternatively or additionally, each of the one or more secondary engagement features may include C-shaped features having a lumen diameter greater than the first diameter of the guidewire and a channel width that is greater than the second diameter of the guidewire.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative medical system;

FIG. 2 is a partial cutaway view of a portion of the illustrative medical system of FIG. 1;

FIG. 3 is a partial cutaway view of a portion of the illustrative medical system of FIG. 1 including a guide catheter;

FIG. 4 is a partial cutaway view of a portion of the illustrative medical system of FIG. 1 including a guide catheter and a working catheter;

FIG. 5 is a partial cutaway view of a portion of the illustrative medical system of FIG. 1 including a multiple diameter guidewire;

FIG. 6 is a partial cutaway view of a portion of the illustrative medical system of FIG. 1 including a guide catheter and a working catheter;

FIG. 7 is a partial cutaway view of a portion of the illustrative medical system of FIG. 1 including a guide catheter and a working catheter;

FIG. 8 is an enlarged view showing the working catheter engaging the guidewire;

FIG. 8A is an enlarged end view of a primary engagement feature of the working catheter;

FIG. 9 is an enlarged view showing the working catheter engaging the guidewire

FIG. 10 is an enlarged view showing the working catheter engaging the guidewire; and

FIG. 11 is an enlarged view showing the working catheter engaging the guidewire.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of medical procedures may utilize a plurality of medical devices such as catheters. A guide catheter may be used to access a particular treatment area, for example. In some instances, the guide catheter may be used to guide a guidewire to the particular treatment area. In some instances, the guidewire may be advanced through the vasculature prior to the guide catheter and perhaps subsequent working catheters being advanced over the guidewire. In some instances, multiple working catheters may be advanced over the guidewire. In some instances, there may be benefits in being able to exchange multiple devices such as catheters by removing one catheter and subsequently advancing another catheter over the guidewire. In some instances, there may be benefits in making this exchange within a saline environment in order to reduce or even eliminate the possibility of air bubbles being dragged into a patient as a medical device such as a catheter is advanced through the vasculature.

An illustrative but non-limiting example of such a procedure is a cardiac valve repair process in which a plurality of working catheters may be used to deliver and deploy repair elements close to the cardiac valve being repaired. A guide catheter may be advanced to a position proximate the cardiac valve in order to guide a guidewire to an appropriate location within the vasculature. Once the guidewire has been appropriately deployed, a series of working catheters may be used to deliver and deploy the repair elements.

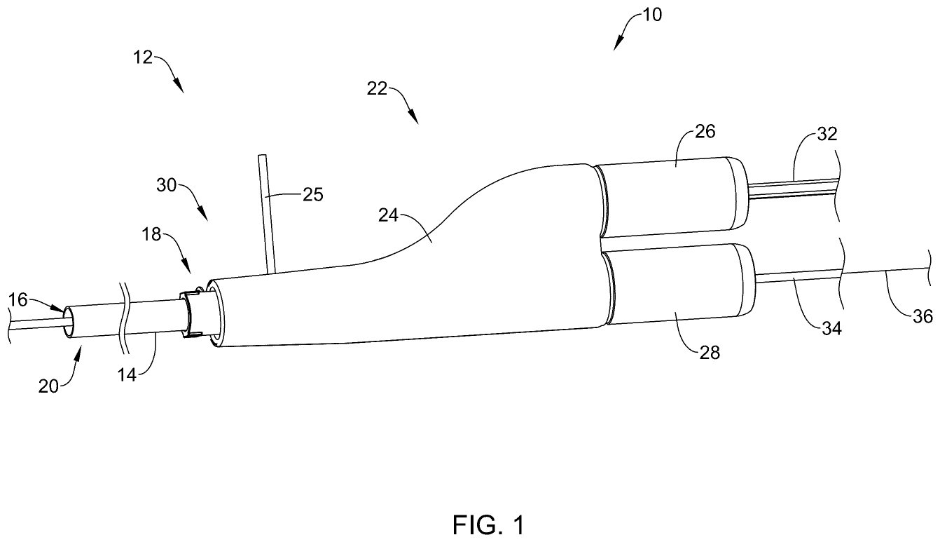

FIG. 1 is a schematic view of an illustrative medical system 10. The illustrative medical system 10 may be utilized in a variety of different medical procedures, and valve repair is simply an example of such a medical procedure. The medical system 10 includes an introducer device 12. The introducer device 12 includes an elongate shaft 14 that defines a shaft lumen 16 extending through the elongate shaft 14. The elongate shaft 14 extends between a proximal region 18 and a distal region 20. A hub 22 is secured to the proximal region 18 of the elongate shaft 14. The hub 22 includes a hub body 24 and a port 25 extending from the hub body 24. In some instances, the port 25 may be used for connecting a source of saline for flushing an interior of the hub body 24.

The introducer device 12 includes a first hemostasis valve 26 and a second hemostasis valve 28. In some instances, as shown, the introducer device 12 may include a third hemostasis valve 30. It will be appreciated that inclusion of the first hemostasis valve 26, the second hemostasis valve 28 and the third hemostasis valve 30 may provide a fluid-tight volume between the first hemostasis valve 26, the second hemostasis valve 28 and the third hemostasis valve 30. Each of the first hemostasis valve 26, the second hemostasis valve 28 and the third hemostasis valve 30 (if included) may be adapted to provide a fluid-tight seal when no devices extend through the hemostasis valve, and to provide a fluid-tight seal against any devices extending through the hemostasis valve.

As shown, a working catheter 32 is extending through the first hemostasis valve 26 and at least partially through the hub 22. A guide catheter 34 is extending through the second hemostasis valve 28 and at least partially through the hub 22. A guidewire 36 is shown within the guide catheter 34, and extends through the hub 22 and through the elongate shaft 14. In some instances, the guide catheter 34 may be steerable, and thus may be used to accurately position the guidewire 36.

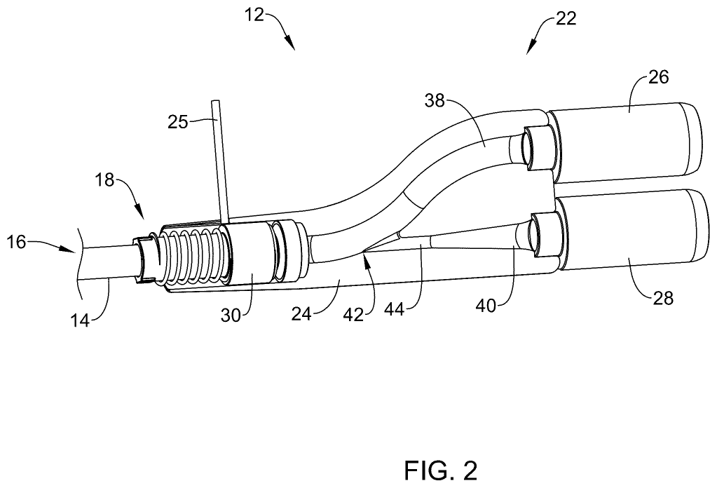

FIG. 2 is a partial cutaway view of the introducer device 12. In FIG. 2, a portion of the hub body 24 has been cutaway to reveal structure within the hub 22. As can be seen, the hub 22 includes a primary channel 38 that extends from the first hemostasis valve 26 (or from a position proximate the first hemostasis valve 26) towards the shaft lumen 16 that extends through the elongate shaft 14 of the introducer device 12. As can be seen, the primary channel 38 curves through an interior of the hub 22 in order to align with both the first hemostasis valve 26 and the shaft lumen 16 (and the intervening third hemostasis valve 30, if present). A secondary channel 40 extends distally from the second hemostasis valve 28 (or from a position proximate the second hemostasis valve 28) and intersects the primary channel 38 at an intersection point 42. In some instances, the primary channel 38 may have a constant or substantially constant (defined as within ten percent) diameter while the secondary channel 40 may have a diameter that tapers proximally to distally. In some instances, the hub 22 may be designed with the first hemostasis valve 26 and the second hemostasis valve 28 in other positions, meaning that the primary channel 38 and the secondary channel 40 may have different geometries. The only caveat is that the introducer device 12 is able to properly position an engagement region of the guidewire 36 so that corresponding engagement features on the working catheter distal end 50 are able to releasably engage and disengage the guidewire 36.

In some instances, the guidewire 36 and the guide catheter 34 may each be independently adapted to be advanced through the second hemostasis valve 28 and through the secondary channel 40 until reaching the primary channel 38 at the intersection point 42. From there, the guidewire 36 and the guide catheter 34 may each be independently adapted to be advanced through the third hemostasis valve 30 (if present) and into the shaft lumen 16 of the elongate shaft 14. The working catheter 32 may be adapted to be advanced through the first hemostasis valve 26 and through the primary channel 38 to a point at which the working catheter 32 is able to releasably engage with the guidewire 36. In some instances, a reduced diameter region 44 of the secondary channel 40 may be dimensioned to have an inner diameter that is about the same or just larger than an outer diameter of the guide catheter 34.

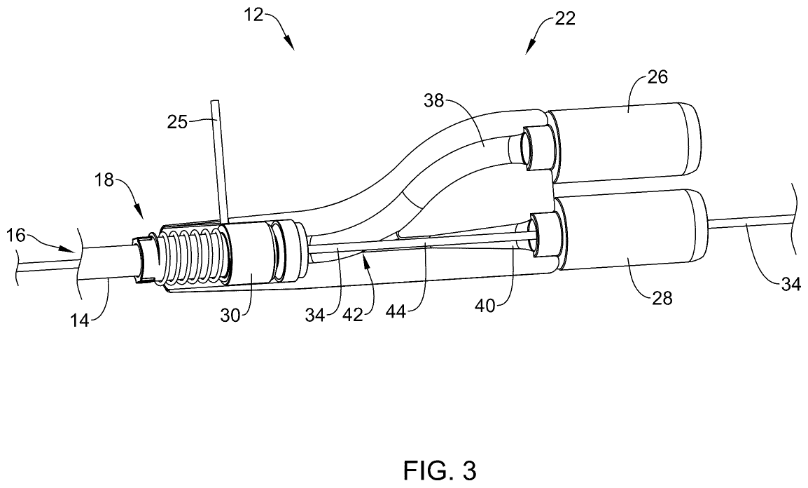

FIG. 3 is a partial cutaway view of the introducer device 12, showing the guide catheter 34 extending through the introducer sheath 12. The guide catheter 34 extends proximally from the second hemostasis valve 28 and extends distally through the elongate shaft 14. In some instances, the guidewire 36 may now be advanced through the guide catheter 34 to an appropriate position near a treatment site. In some instances, the guidewire 36 may have been advanced through introducer device 12 to the appropriate treatment position near the treatment site prior to advancing the guide catheter 34. It will be appreciated that the guide catheter 34 has an outer diameter that can be seen as being about equal to the diameter of the reduced diameter region 44 of the secondary channel 40.

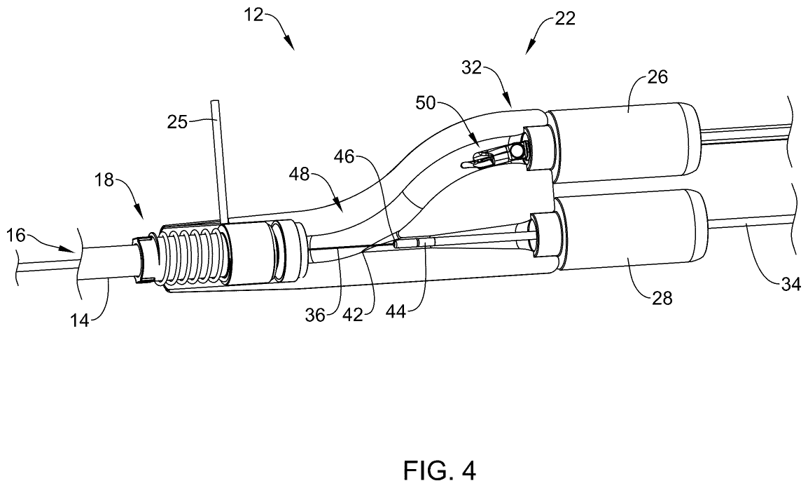

FIG. 4 is a partial cutaway view of the introducer device 12, showing the guide catheter 34 withdrawn proximally such that a distal end 46 of the guide catheter 34 is disposed near the intersection point 42. In some instances, positioning the guide catheter 34 in this position means that the guide catheter 34 helps to center and thus locate the guidewire 36. In some instances, inclusion of the third hemostasis valve 30 also helps to center and thus locate the guidewire 36. In some instances, centering and locating the guidewire 36 within a distal region 48 of the primary channel 38 is beneficial in getting the working catheter 32 to releasably engage the guidewire 36, as will be discussed. The working catheter 32 includes a distal region 50 that is adapted to releasably engage the guidewire 36, as will be described.

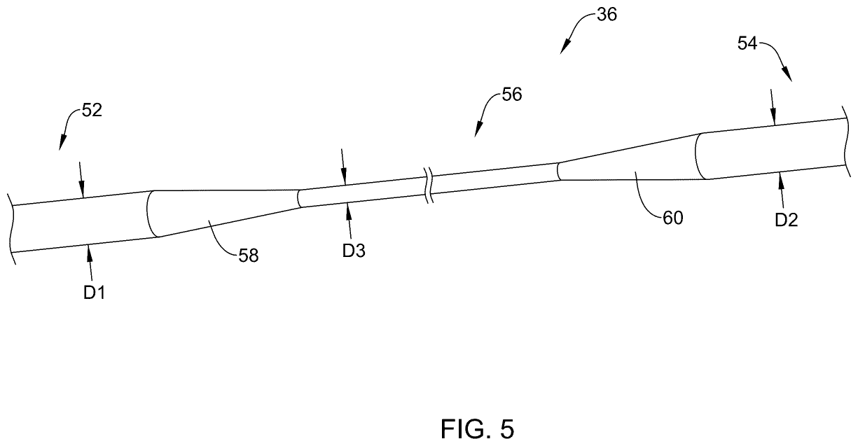

FIG. 5 is a schematic view of the guidewire 36. In some instances, the guidewire 36 extends from a distal region 52 to a proximal region 54. In some instances, the distal region 52 of the guidewire 36 has a diameter D₁ and the proximal region 54 of the guidewire has a diameter D₂. In some instances, the diameter D₁ is equal to the diameter D₂. In some instances, as shown, the diameter D₁ may be smaller than the diameter D₂. In some instances, the guidewire 36 includes an engagement region 56 that is located between the distal region 52 and the proximal region 54. The engagement region 56 may have a diameter D₃ that is less than either the diameter D₁ or the diameter D₂. In some instances, the guidewire 36 may include a distal tapered region 58 that provides a smooth and gradual change in diameter from the diameter D₁ to the diameter D₃. In some instances, the guidewire 36 may include a proximal tapered region 60 that provides a smooth and gradual change in diameter from the diameter D₂ to the diameter D₃.

In some instances, D₁ and D₂ are equal to each other, and are defined at least in part upon an overall size of the guidewire 36. As an example, the guidewire 36 may be referred to as an 0.035 inch guidewire, meaning that both D₁ and D₂ are equal to 0.035 inches. In some instances, D₁ and D₂ may vary, depending on the particular guidewire, but are equal to each other. Various size guidewires may be used, depending at least in part upon the dimensions of the vasculature through which the guidewire will be expected to pass and the dimensions and other requirements of the guide catheter and working catheters to be advanced over the guidewire. In some instances, it is contemplated that D₁ and D₂ may vary from each other. As an example, D₁ may be smaller than D₂ in situations in which having a smaller diameter distal region 52 may be beneficial.

For the engagement region 56, the diameter D₃ may be considered as being a fraction of either D₁ or D₂. As an example, D₃ may be in a range of about 20 to 80 percent of either D₁ or D₂. As another example, D₃ may be in a range of about 30 to 70 percent of either D₁ or D₂, or perhaps a range of about 40 to 60 percent of either D₁ or D₂. In some instances, the guidewire 36 may be a constant diameter guidewire in which the engagement region 56 has been ground down to achieve the diameter D₃. As will be discussed, the engagement region 56 allows the working catheter 32 to releasably engage and disengage the guidewire 36.

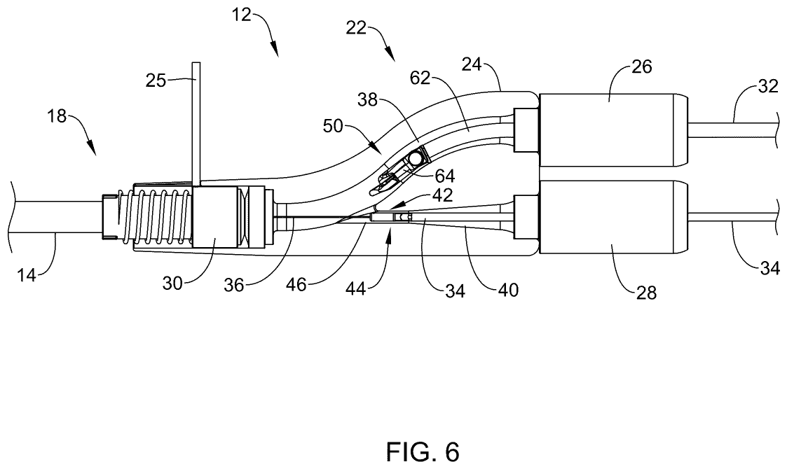

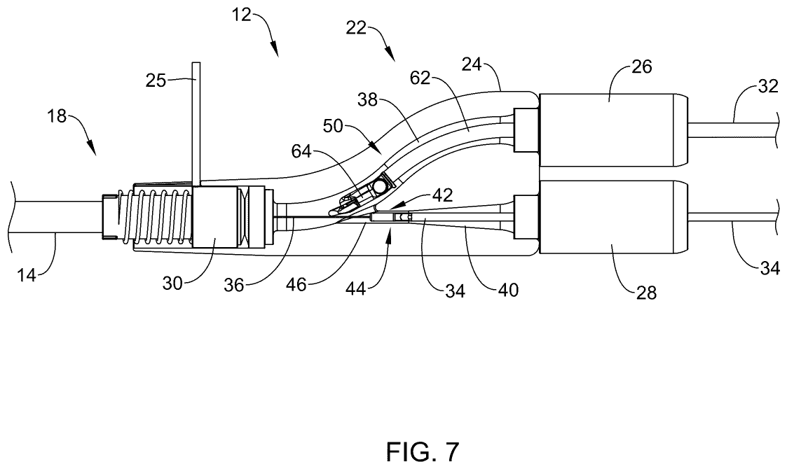

FIG. 6 is a partial cutaway view of the introducer device 12, again showing the guide catheter 34 withdrawn proximally such that a distal end 46 of the guide catheter 34 is disposed near the intersection point 42. At the same time, the working catheter 32 can be seen as advancing distally through the primary channel 38. As can be seen, the working catheter 32 includes a catheter shaft 62 and a distal body 64 secured relative to the catheter shaft 62 within the distal region 50 of the working catheter 32. In FIG. 7, the working catheter 32 is seen as approaching a position in which engagement features within the distal body 64 allow the working catheter 32 to releasably engage and disengage the guidewire 36. In some instances, the engagement features are adapted to engage with the reduced diameter engagement portion 56 of the guidewire 36. In some instances, whether the engagement features remain engaged with the guidewire 36 or become disengaged from the guidewire 36 may be influenced by moving the guidewire 36 proximally or distally to position the reduced diameter engagement portion 56 as desired relative to the engagement features.

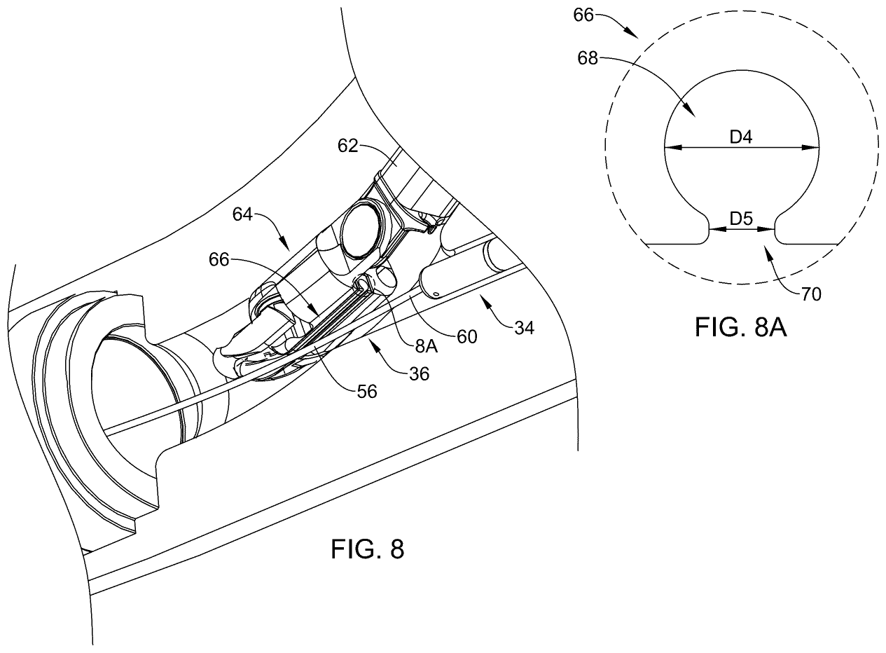

FIG. 8 is an enlarged view of a lower portion of the working catheter 32, including the distal body 64. An underside (in the illustrated orientation) of the distal body 64 includes a primary engagement feature 66 that is adapted to releasably engage the guidewire 36. In some instances, the primary engagement feature 66 may be considered as having a C-shaped cross-sectional profile. The primary engagement feature 66 includes a body lumen 68 having a body lumen diameter indicated by D₄. The primary engagement feature 66 also includes a channel 70 that is operably coupled with the body lumen 68. The channel 70 has a channel width indicated by D₅ that is less than the body lumen diameter D₄. In some instances, the channel width D₅ is less than the diameter D₁ or the diameter D₂ of the guidewire 36, meaning that when either the distal region 52 or the proximal region 54 of the guidewire 36 extends through the primary engagement feature 66, the guidewire 36 is held within the body lumen 68. In some instances, the channel width D₅ is greater than the diameter D₃, meaning that the engagement region 56 of the guidewire 36 is able to slip in and out of the body lumen 68 through the channel 70. The body lumen diameter D₄ is greater than either D₁ or D₂, meaning that the guidewire 36 is able to translate within the body lumen 68.

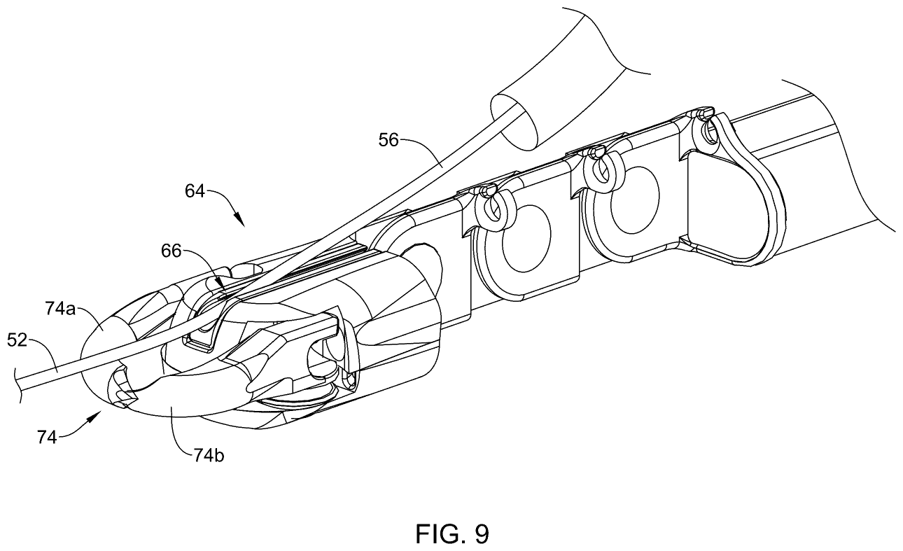

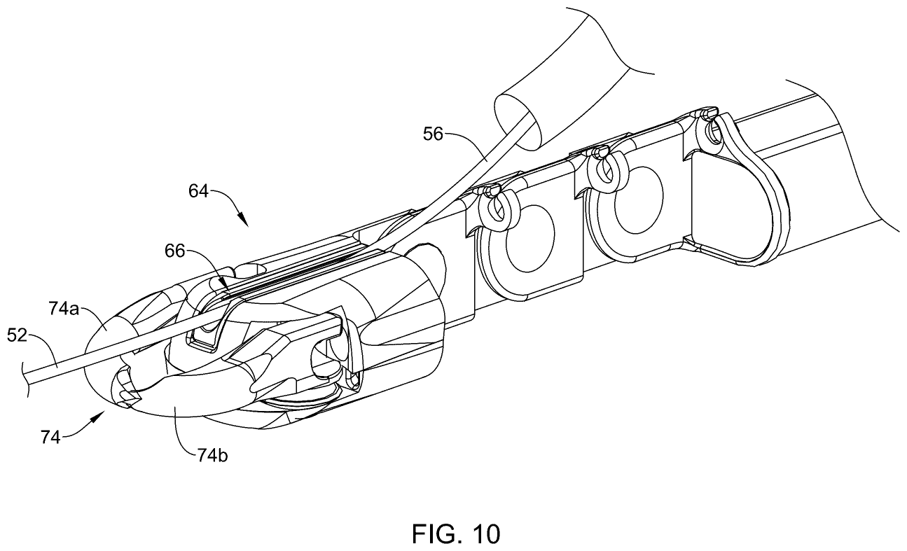

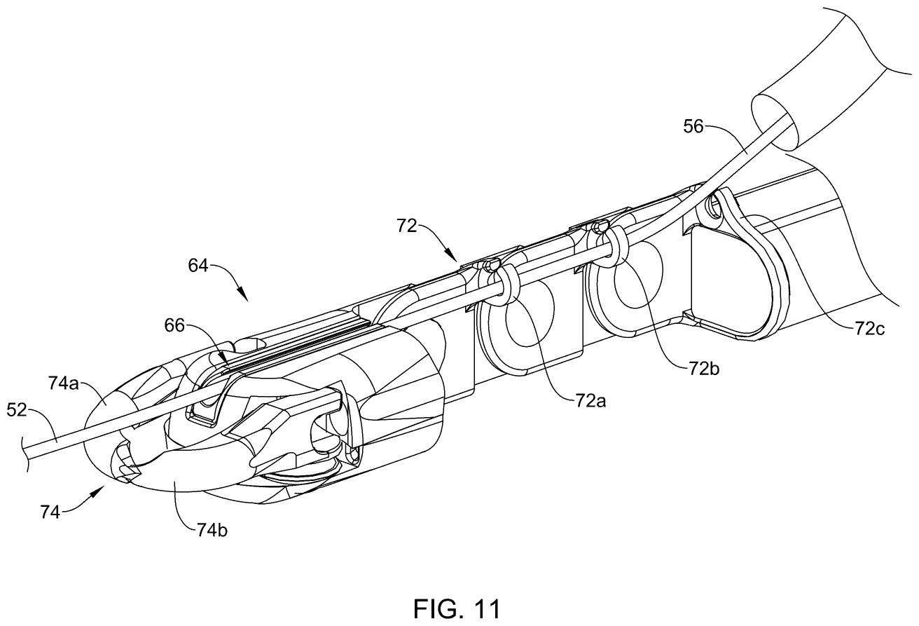

FIGS. 9 through 11 provide additional details regarding the distal body 64. It will be appreciated that in FIGS. 9-11, the distal body 64 is shown in an inverted configuration for clarity. FIGS. 9 through 11 provide a sequence showing how the engagement region 56 of the guidewire 36 increasingly engages the primary engagement feature 66 as the working catheter 32 moves distally through the primary channel 38. In FIG. 9, the engagement region 56 of the guidewire 36 has partially engaged the primary engagement feature 66. In FIG. 10, the engagement region 56 of the guidewire 36 has almost completely engaged the primary engagement feature 66. In FIG. 11, the engagement region 56 of the guidewire 36 has completely engaged the primary engagement feature 66 and is in the process of engaging one or more secondary engagement features 72, individually labeled as 72a, 72b and 72c. In some instances, the distal body 64 may not include any secondary engagement features 72, or may only include one or two secondary engagement features 72. In some instances, the distal body 64 may include four, five or more secondary engagement features. As can be seen, each of the secondary engagement features 72 are C-shaped, and have a lumen diameter greater than either D₁ or D₂, and a channel width that is larger than D₃ (yet smaller than either D₁ or D₂).

In some instances, the distal body 64 may include a working feature 74. As shown, the working feature 74 includes a pair of pincers labeled 74a and 74b. This is merely an example, as the working catheter 32 may include any of a variety of different working features 74. In some instances, the pincers 74a and 74b may be used for holding and delivering elements useful in repairing a cardiac valve, for example.

Once the guidewire 36 has been advanced through the vasculature to a desired treatment location, the guide catheter 34 may be withdrawn proximally to a position in which the distal end 46 of the guide catheter 34 is proximate the intersection point 42 between the primary channel 38 and the secondary channel 40, a working catheter such as the working catheter 32 may be advanced distally through the primary channel 38 until the primary engagement feature 66 (and secondary engagement features 72, if present) engage the engagement region 56 of the guidewire 36. In some instances, this may include translating the guidewire 36 distally or proximally in order to properly position the engagement region 56 of the guidewire 36. Once engaged, the working catheter 32 may be moved distally (or the guidewire 36 may be moved proximally) in order to dispose the distal region 52 of the guidewire 36 within the primary engagement feature 66 (and optionally within the secondary engagement features 72, if present).

Once the working catheter 32 is no longer needed, the working catheter 32 may be withdrawn proximally until the distal body 64 is positioned within the primary channel 38. The guidewire 36 may be translated to position the engagement region 56 of the guidewire 36 within the primary engagement feature 66 (and optionally within the secondary engagement features 72, if present). With the guide catheter 34 positioned with its distal end 46 proximate the intersection point 42), continuing to withdraw the working catheter 32 proximally will cause the engagement region 56 of the guidewire 36 to disengage from the primary engagement feature 66 and optionally the secondary engagement features 72, if present.

In some instances, there may be a desire to use one or more additional working catheters. It will be appreciated that each of the one or more additional working catheters may be advanced through the introducer device 12 in a manner similar to that described with respect to the working catheter 32. Because of the hemostasis valves 26, 28 and 30, each additional working catheter will enter the hub 22 via the first hemostasis valve 26 into a saline field prior to engaging and following the guidewire 36 into the patient. This allows any air bubbles entrained within the working catheter being introduced to escape into the saline within the saline field.

The materials that can be used for the various components of the medical devices described herein may include those commonly associated with medical devices. The medical devices described herein, as well as individual components thereof, be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids in determining a location of a medical device that includes a radiopaque material. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into various medical devices to achieve the same result.

The medical devices described herein, as well as portions and components thereof, may be made of the same material along its length, or in some embodiments, can include portions or sections made of different materials. In some embodiments, materials may be chosen to impart varying flexibility and stiffness characteristics to different portions. For example, different portions of a component, such as a proximal section and a distal section, may be formed of different materials, for example, materials having different moduli of elasticity, resulting in a difference in flexibility. In some embodiments, the material used to construct a proximal section may be relatively stiff for pushability and torqueability, and the material used to construct a distal section may be relatively flexible by comparison for better lateral trackability and steerability. For example, a proximal section may be formed of straightened 304v stainless steel wire or ribbon and a distal section may be formed of a straightened super elastic or linear elastic alloy, for example a nickel-titanium alloy wire or ribbon.

In embodiments where different portions of the medical devices described herein are made of different materials, the different portions can be connected using a suitable connecting technique and/or with a connector. For example, the different portions may be connected using welding (including laser welding), soldering, brazing, adhesive, or the like, or combinations thereof. These techniques can be utilized regardless of whether or not a connector is utilized. An example of a connector is a structure such as a hypotube or a coiled wire which has an inside diameter sized appropriately to receive and connect to the ends of the proximal portion and the distal portion.

A sheath or covering (not shown) may be disposed over portions or all of the medical devices described herein. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical devices described herein may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the medical devices described herein. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluorocthylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical system, comprising:
an introducer device including an elongate shaft defining a shaft lumen therethrough, the elongate shaft including a proximal region, and a proximal hub secured relative to the proximal region of the elongate shaft, the proximal hub including:
a primary channel extending through the proximal hub and fluidly coupled with the shaft lumen;
a secondary channel extending through the proximal hub and fluidly coupled with the shaft lumen;
a guide catheter adapted to be advanced through the secondary channel and into the shaft lumen;
a guidewire adapted to be advanced through the secondary channel; and
a working catheter adapted to be advanced through the primary channel and into the shaft lumen when the guide catheter has been withdrawn proximally such that a distal end of the guide catheter remains within the secondary channel, the working catheter including a catheter shaft including a distal region, the distal region of the working catheter adapted to releasably engage the guidewire.

2. The medical system of claim 1, further comprising:
a first hemostasis valve fluidly coupled with the primary channel; and
a second hemostasis valve fluidly coupled with the secondary channel.

3. The medical system of claim 2, further comprising a third hemostasis valve disposed between the proximal hub and the elongate shaft, thereby providing a fluid tight volume within the primary channel and the secondary channel.

4. The medical system of claim 1, wherein the guidewire has a first diameter over the guidewire outside of an engagement region, and a second diameter less than the first diameter within the engagement region.

5. The medical system of claim 4, wherein the distal region of the working catheter includes a distal body defining a primary engagement feature adapted to releasably engage the guidewire.

6. The medical system of claim 5, wherein the primary engagement feature comprises:
a body lumen having a body lumen diameter; and
a channel operably coupled with the body lumen, the channel having a channel width less than the body lumen diameter.

7. The medical system of claim 6, wherein the channel width is less than the first diameter of the guidewire and greater than the second diameter of the guidewire, and the body lumen diameter is greater than the first diameter of the guidewire.

8. The medical system of claim 5, wherein the engagement feature further comprises one or more secondary engagement features disposed proximally of the primary engagement feature.

9. The medical system of claim 8, wherein each of the one or more secondary engagement features comprise C-shaped features having a lumen diameter greater than the first diameter of the guidewire and a channel width that is greater than the second diameter of the guidewire.

10. The medical system of claim 1, wherein the primary channel is curved and the secondary channel is straight and intersects the primary channel.

11. An introducer device for use with a guide catheter and a working catheter, the introducer device comprising:
an elongate shaft defining a shaft lumen therethrough, the elongate shaft including a proximal region; and
a hub secured relative to the proximal region of the elongate shaft, the hub including:
a first hemostasis valve disposed at a proximal end of the hub;
a second hemostasis valve disposed at the proximal end of the hub, the second hemostasis valve parallel with but spaced from the first hemostasis valve;
a primary channel extending distally through the hub from the first hemostasis valve to the shaft lumen; and
a secondary channel extending distally through the hub from the second hemostasis valve and fluidly coupled with the primary channel.

12. The introducer device of claim 11, wherein the primary channel curves through the hub from the first hemostasis valve towards the shaft lumen.

13. The introducer device of claim 11, wherein the secondary channel extends linearly through the hub from the second hemostasis valve to an intersection between the primary channel and the secondary channel.

14. The introducer device of claim 13, wherein a diameter of the secondary channel narrows from proximal to distal.

15. The introducer device of claim 11, further comprising a third hemostasis valve fluidly coupled between the primary channel and the shaft lumen, thereby providing a fluid-tight volume between the first hemostasis valve, the second hemostasis valve and the third hemostasis valve.

16. A working catheter adapted to be used in combination with an introducer device having a bifurcated hub, the bifurcated hub defining a first passageway adapted to accommodate the working catheter and a second passageway adapted to accommodate a guide catheter and a guidewire, the second passageway intersecting the first passageway, the guidewire having a first diameter outside of an engagement region and a second diameter less than the first diameter within the engagement region, the working catheter comprising:
an elongate catheter shaft extending from a proximal region to a distal region;
a working member secured within the distal region, the working member comprising:
a primary engagement feature that is adapted to releasably secure the body member to a guidewire; and
one or more secondary engagement features that are each adapted to releasably secure the body member to the guidewire.

17. The working catheter of claim 16, wherein the working member further comprises a working feature.

18. The working catheter of claim 16, wherein the primary engagement feature comprises:
a body lumen having a body lumen diameter that is greater than the first diameter of the guidewire; and
a channel operably coupled with the body lumen, the channel having a channel width that is less than the first diameter of the guidewire and greater than the second diameter of the guidewire.

19. The working catheter of claim 18, wherein the channel width is less than a first diameter of the guidewire and greater than a second diameter of the guidewire, and the body lumen diameter is greater than the first diameter of the guidewire.

20. The working catheter of claim 19, wherein each of the one or more secondary engagement features comprise C-shaped features having a lumen diameter greater than the first diameter of the guidewire and a channel width that is greater than the second diameter of the guidewire.
